(19) United States
(12) Patent Application Publication
(10) Pub. No.: US 2015/0150979 A1
(43) Pub. Date: Jun. 4, 2015

(54) PHARMACEUTICAL FORMULATION

(71) Applicant: UCB PHARMA, S.A., BRUSSELS (BE)

(72) Inventors: Andrew Jeffrey Yates, Slough (GB); James Gregory Clipstone, Slough (GB)

(21) Appl. No.: 14/406,758

(22) PCT Filed: Jun. 20, 2013
(86) PCT No.: PCT/EP2013/062898

§ 371 Date: Dec. 10, 2014

Related U.S. Application Data
(60) Provisional application No. 61/662,621, filed on Jun. 21, 2012.

(51) Int. Cl. A61K 47/12; A61K 47/26; A61K 47/18; A61K 47/02; A61K 39/395; A61K 9/00

(52) U.S. Cl. CPC A61K 47/12; A61K 39/3955; A61K 9/0019; A61K 47/183; A61K 47/02; A61K 47/26

(57) ABSTRACT

The present invention relates to methods and means for reducing the viscosity of a pharmaceutical formulation comprising an antibody or other therapeutic protein at a high concentration. The present invention provides a liquid pharmaceutical formulation comprising an antibody at a high concentration with reduced viscosity that does not impede processing or injection of the pharmaceutical formulation.

### FIELD OF THE INVENTION

The present invention is in the field of pharmaceutical formulations. More specifically, it relates to a pharmaceutical formulation comprising a protein such as an antibody.

### THE INVENTION

Antibodies, as other protein therapeutics are large and complex molecules and are inherently instable both, chemically and physically potentially resulting in a reduction or loss of activity. Typical chemical instability may result in deamidation, hydrolysis, oxidation, beta-elimination or disulfide exchanges. Physical instability can result in denaturation, aggregation or precipitation.

Therefore, for storage, transport, handling and administration pharmaceutical formulations of antibodies and other proteins have to minimize any of the above phenomena. Antibodies can be formulated in freeze-dried; i.e. lyophilized, form for reconstitution in a solvent shortly before administration, or antibodies can be formulated in liquid form, such as in an aqueous solution. Freeze-dried formulations of antibodies tend to be more stable as water is either a reactant or as a solvent facilitates the transfer of reactants and is thus critical to many routes of chemical degradation that lead to protein instability (Andya et al., 2003). Despite the tendency to be less stable interest has recently focused on liquid formulations of antibodies and other proteins as these are easier and more convenient for the patient and the healthcare professional to handle and administer in comparison with freeze-dried formulations. Liquid formulations do not need to be reconstituted and can be administered with minimal preparation. There is therefore a need to develop stable liquid formulations of antibodies and other proteins. The stabilization of proteins in liquid formulations to avoid or minimize unwanted reactions such as aggregation, precipitation or degradation remains a particular challenge.

Aggregation is particular problem. Individual protein molecules stick physically together resulting, for example, in the formation of insoluble matter or precipitate, which may no longer be active and even cause undesired immunological reactions upon administration. Additionally, a major problem caused by the aggregate formation is that during the administration the pharmaceutical formulation may block syringes or pumps.

Conveniently, liquid pharmaceutical formulations of antibodies and other protein therapeutics should be long-term stable, and minimize the above reactions in order to contain the correct amount of pharmaceutical ingredient in active form.

Frequently antibodies or other therapeutic proteins have to be administered in high doses to be therapeutically effective. A convenient way to administer antibodies or proteins is through subcutaneous injection. Pharmaceutical formulations of antibodies or other therapeutic proteins for subcutaneous injection pose a particular challenge as the volume of liquid that can be injected per injection into a site is limited, generally to about 1 to 2 ml per injection, and multiple injections per dose are inconvenient for the subject receiving the injection thereby causing frequently lack of compliance and subsequently incorrect dosing. Therefore pharmaceutical formulations of antibodies or other therapeutic proteins for subcutaneous injection frequently require a high concentration of active ingredient.

Increasing protein concentration often negatively impacts protein aggregation, solubility, stability, and viscosity. High concentration of antibodies in pharmaceutically formulations typically leads to high viscosity (Liu et al., 2005). The factors leading to high viscosity in the concentrated solution are not well understood but are considered to be the impacted by molecular crowding as the proportion of solvent drops and by direct interactions among the proteins. Remarkably, molecule-specific effects have been reported such that solutions of structurally very similar proteins can have different viscosities at the same concentration (Galush et al., 2012).

A high viscosity of a liquid pharmaceutical formulation poses problems including with regard to the processing of the pharmaceutical formulation as well as during administration. Processing involves the filling of the pharmaceutical formulation into vial or syringes or other containers for storage, transport or administration. Highly viscous liquid formulations may also cause problems when administered by injection. Highly viscous liquid formulations require a high pressure when injected through a needle. Highly viscous liquid formulations also require more time to be injected causing discomfort to the patient.

Thus, there is a need for a liquid pharmaceutical formulation comprising a protein, in particular an antibody, at high concentration which is stable and substantially free of aggregates having a viscosity that allows injection with a needle either manually or through a device. Generally, pharmaceutical formulations comprising antibody or other therapeutic protein at a concentration of at least 100 mg/ml are considered high concentration formulations.

A strategy for reducing the viscosity of a high concentration protein formulation known in the art is based on the addition of ions or salts thereof which reduce the self-association of proteins. Chaotropic ions, such as for example, HCO₃⁻, Cl⁻, K⁺ ions, destabilize hydrophobic interactions and are preferred. Kosmotropic ions, such as for example, Mg²⁺, Ca²⁺, Na⁺ ions, stabilize hydrophobic interactions in solution work as well but are generally less preferred (Liu, Nguyen, Andya, & Shire, 2005). Ions can, however, have an effect on the conformational stability of the protein or antibody in solution and sometimes even lead to increased aggregation (He et al., 2010).

U.S. Pat. No. 7,666,413 relates to a method of reducing viscosity of high concentration protein formulations involving the increase of total ionic strength or the alteration of the pH. It is proposed in U.S. Pat. No. 7,666,413 to increase the ionic strength through either the addition of salts or buffers. Data are disclosed which show that in a liquid formulation comprising an antibody at a concentration of 80 mg/ml the addition of histidine or succinate results in a much more enhanced reduction of viscosity than the addition of acetate.

WO 02/096457 relates to stable liquid formulations comprising at least one acidic component. Liquid high concentration antibody formulations are disclosed that comprise between 0 and 17.3 mM acetic acid. Data are disclosed showing that the reduction of the concentration of acetic acid, e.g. from 17.3 mM to 8.7 mM, resulted in reduced viscosity.

WO 2007/076062 relates to protein formulations and methods for reducing the viscosity of a protein formulation comprising adding calcium chloride or magnesium chloride.

In another approach sugars such as trehalose, sucrose, sorbitol, glucose, fructose, xylose or galactose have been used in liquid formulations of protein or antibody to reduce viscosity (He et al., 2011).

However, development of liquid formulations suitable for routine therapeutic use, in particular for subcutaneous administration, comprising antibody substantially above 100 mg/ml, such as e.g. 150 mg/ml, 200 mg/ml or even 300 mg/ml, have faced particular challenges.

In addition to high viscosity high concentration protein or antibody formulations may exhibit undesirable opalescence (Sukumar et al., 2004). Opalescence can give rise to a potential safety issue because an opalescent solution may be confused with a turbid solution, which can result from protein aggregation or other particulate formation. It is also challenging to develop a placebo formulation for clinical studies that match the opalescence of the original formulation.

The murine monoclonal antibody, LL2 (originally named EPB-2), is a B-cell (CD22)-specific IgG₂ₐ monoclonal antibody generated against Raji Burkitt lymphoma cells, and found to be highly selective for normal B-cells and B-cell tumors. A humanized IgG_{1(K)} form of the murine LL2, was developed for clinical use and named epratuzumab (hLL2) (Leung et al., 1995). The construct encoding epratuzumab was created by grafting the complementarity-determining regions (CDR) of the murine parental origin antibody in a human IgG₁ genetic backbone. Epratuzumab has been tested in clinical development for the treatment of systemic lupus erythematosus (SLE) and other autoimmune diseases as well as cancer. Epratuzumab has been shown to be particularly effective when given at a dose of 400 to 800 mg once every week for 4 times in a treatment cycle of 12 weeks or 1000 to 1400 mg once every other week for 2 times in a treatment cycle of 12 weeks (WO 2011/032633). Thus, a useful dosage regimen for epratuzumab requires the administration of between 400 to 800 mg or even 1000 to 1200 mg epratuzumab at a single time point. Currently such amounts of epratuzumab are administered by way of intravenous infusion. Intravenous infusion requires the intervention of a healthcare professional and can often only be performed in an hospital or infusion center. Subcutaneous injection does not generally require the intervention of a healthcare professional and can frequently be performed at home either by the subject receiving the injection itself or another person such a cohabitant or friend. Subcutaneous injection is thereby more patient friendly and increases compliance with the prescribed dosage regimen. Repeated subcutaneous injections of a medicament in order to administer the prescribed amount of medicament is inconvenient for the individual requiring the medicament and generally not well tolerated leading to lack of compliance.

There is therefore a need for a liquid pharmaceutical formulation comprising epratuzumab in high concentration which can be administered by subcutaneous injection, preferably by a single injection.

### SUMMARY OF THE INVENTION

High concentration of proteins such as antibodies in solution has been observed to result generally in a high viscosity. The viscosity of a solution containing a monoclonal antibody increases exponentially with elevating concentration of antibody (FIG. 1).

It has now been found by the present inventors that the addition of acetate has a surprising effect on reducing the viscosity of a pharmaceutical formulation comprising a therapeutic proteins such as an antibody at high concentration. Surprisingly, the increase of ion concentration such as for example through addition of sodium chloride only resulted in very moderate reduction of the viscosity (FIG. 2) whereas the addition of acetate resulted a very substantial reduction in viscosity (FIG. 3).

Accordingly, the invention relates to a methods and means for reducing the viscosity of a pharmaceutical formulation comprising an antibody or other therapeutic protein at a high concentration. The present invention provides a liquid pharmaceutical formulation comprising an antibody at a high concentration with reduced viscosity that does not impede processing or injection of the pharmaceutical formulation.

In one aspect the invention provides a stable liquid pharmaceutical formulation comprising an antibody or other protein at a high concentration with reduced viscosity.

In one embodiment of this aspect of the invention the pharmaceutical formulation comprises an antibody or other protein at a concentration of at least 220 mg/ml.

In another embodiment of this aspect of the invention the pharmaceutical formulation comprises an antibody or other protein at a concentration of at least 250 mg/ml.

In another embodiment of this aspect of the invention the pharmaceutical formulation comprises an antibody or other protein at a concentration of at least 270 mg/ml.

In another embodiment of this aspect of the invention the pharmaceutical formulation comprises an antibody or other protein at a concentration of at least 300 mg/ml.

In another embodiment of this aspect of the invention the pharmaceutical formulation according to any of the embodiments of the invention comprises an antibody or other protein at a concentration of equal or less than 400 mg/ml.

In another embodiment of this aspect of the invention the pharmaceutical formulation according to any of the embodiments of the invention comprises an antibody or other protein at a concentration of equal or less than 350 mg/ml.

In another embodiment of this aspect of the invention the pharmaceutical formulation according to any of the embodiments of the invention comprises acetate at a concentration of at least 40 mM.

In another embodiment of this aspect of the invention the pharmaceutical formulation according to any of the embodiments of the invention comprises acetate at a concentration of at least 55 mM.

In another embodiment of this aspect of the invention the pharmaceutical formulation according to any of the embodiments of the invention comprises acetate at a concentration of at least 90 mM.

In another embodiment of this aspect of the invention the pharmaceutical formulation according to any of the embodiments of the invention comprises acetate at a concentration of 40 to 100 mM.

In another embodiment of this aspect of the invention the pharmaceutical formulation according to any of the embodiments of the invention has an osmolality of equal or less than 450 mOsm/kg, preferably equal or less than 410 mOsm/kg, more preferably equal or less 370 mOsm/kg, more preferably equal or less than 310 mOsm/kg, and most preferably from 275 to 310 mOsm/kg.

In another embodiment of this aspect of the invention the pharmaceutical formulation according to any of the embodiments of the invention has a viscosity of equal or less than 110 mPa s.

### BRIEF DESCRIPTION OF THE DRAWINGS

FIG. 1 shows the viscosity (cP) of two liquid formulations plotted against the concentration of the monoclonal antibody epratuzumab in the formulation. The formulations contain 60 mM NaOAc, 0.01% Polysorbate 80, 220 mM and 420 mM glycine, respectively at pH 5.0. The concentration of glycine has essentially no impact on the viscosity.

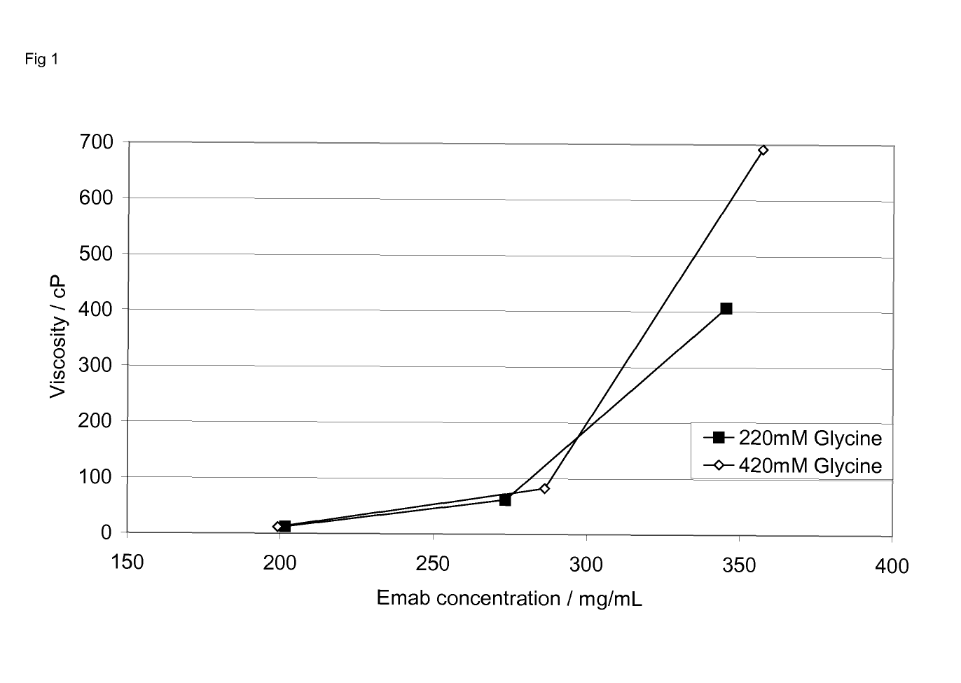

FIG. 2 shows the viscosity (cP) plotted against the NaCl concentration of a pharmaceutical formulation containing epratuzumab at 300 mg/mL, 80 mM sodium acetate (NaOAc), 220 mM glycine and 0.01% Polysorbate 80, pH 5.0.

FIG. 3 shows the viscosity (cP) plotted against the NaOAc concentration of a pharmaceutical formulation containing epratuzumab at 300 mg/mL 0 mM NaCl, 220 mM glycine and 0.01% Polysorbate 80, pH 5.0.

FIG. 4 shows in a three dimensional diagram the relationship between viscosity (measured in cP), acetate concentration and concentration of the monoclonal antibody epratuzumab in a liquid formulation containing 0 mM NaCl, 220 mM glycine, 0.01% Polysorbate 80 and having pH 5.0.

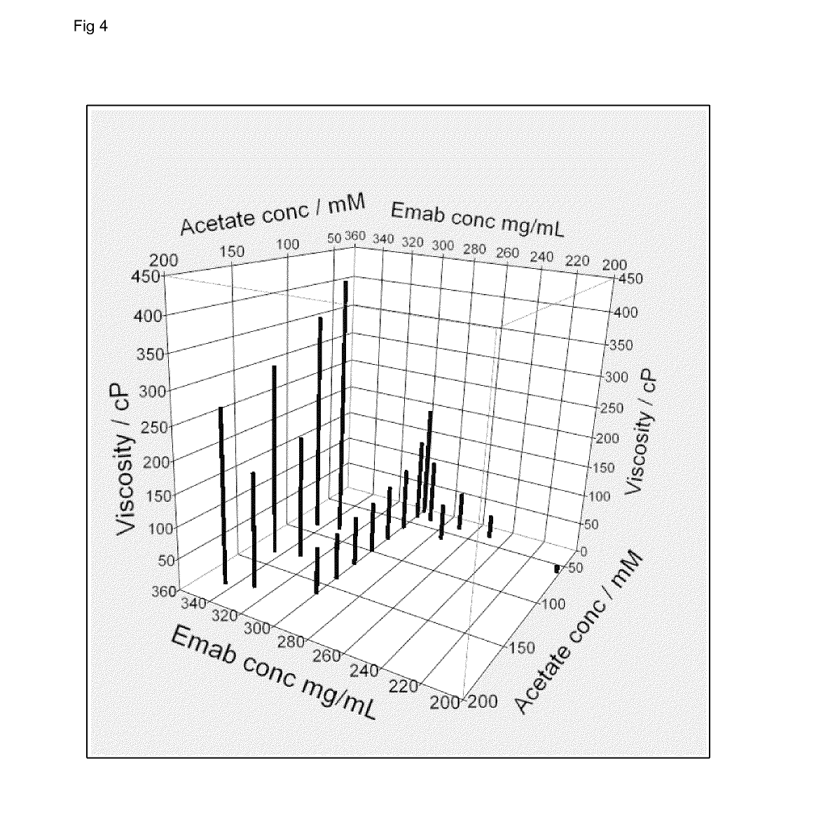

FIG. 5 shows the impact of the NaOAc concentration (mM) on the modeled osmolality (mOsm/L) of liquid formulations containing the monoclonal antibody epratuzumab at different concentrations and 0 mM NaCl, 220 mM glycine, 0.01% Polysorbate 80 and having pH 5.0.

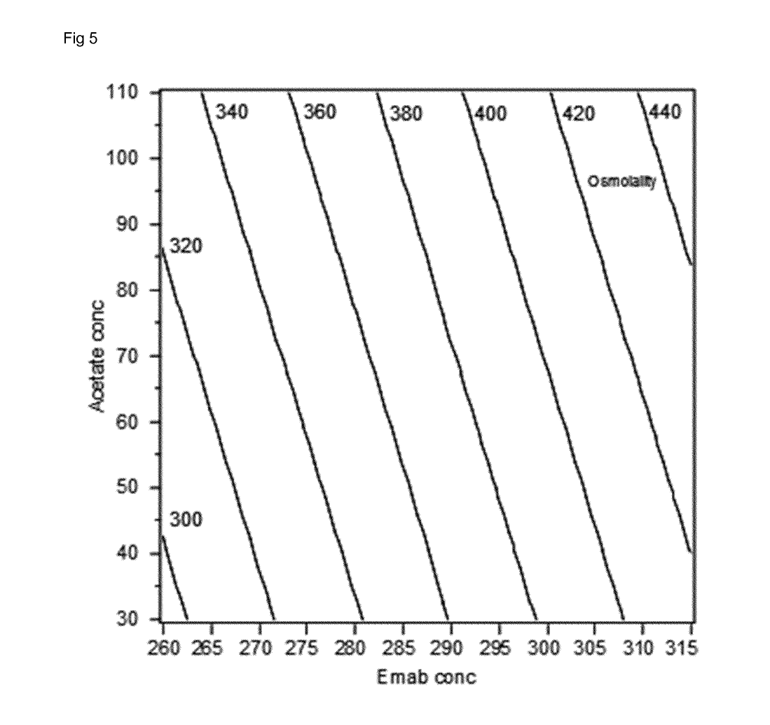

FIG. 6 shows the level of high molecular weight (HMW) and low molecular weight (LMW) species per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively as a function of freeze thaw cycles. Data were measured by size exclusion chromatography (SEC).

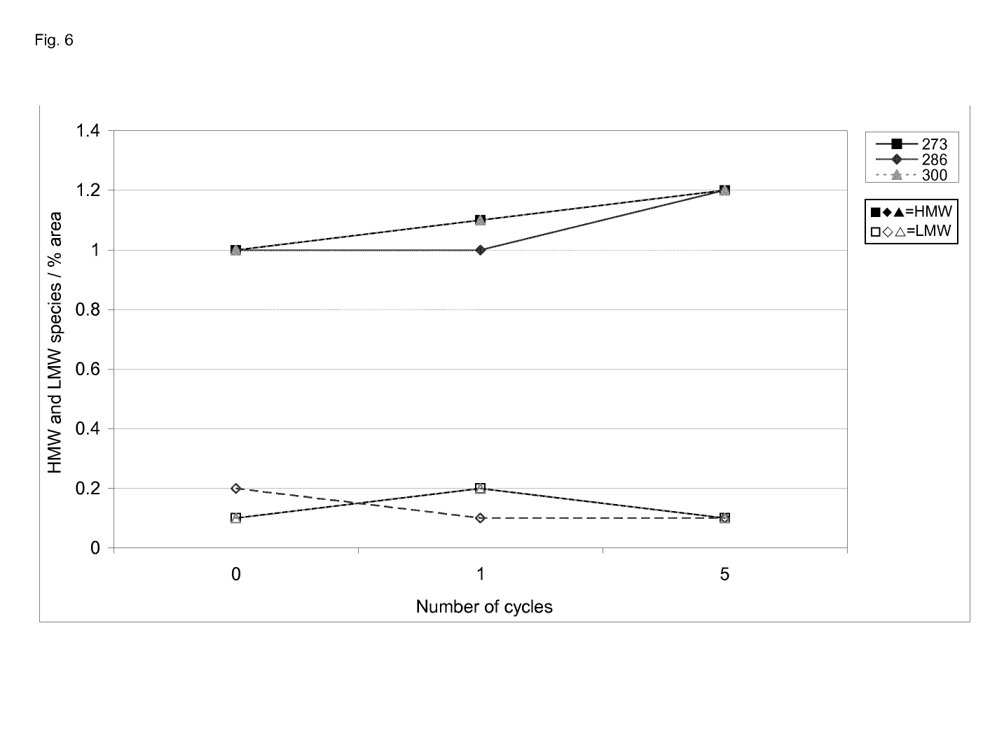

FIG. 7 shows the level of HMW and LMW species per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 5° C. as a function of time. Data were measured by size exclusion chromatography (SEC).

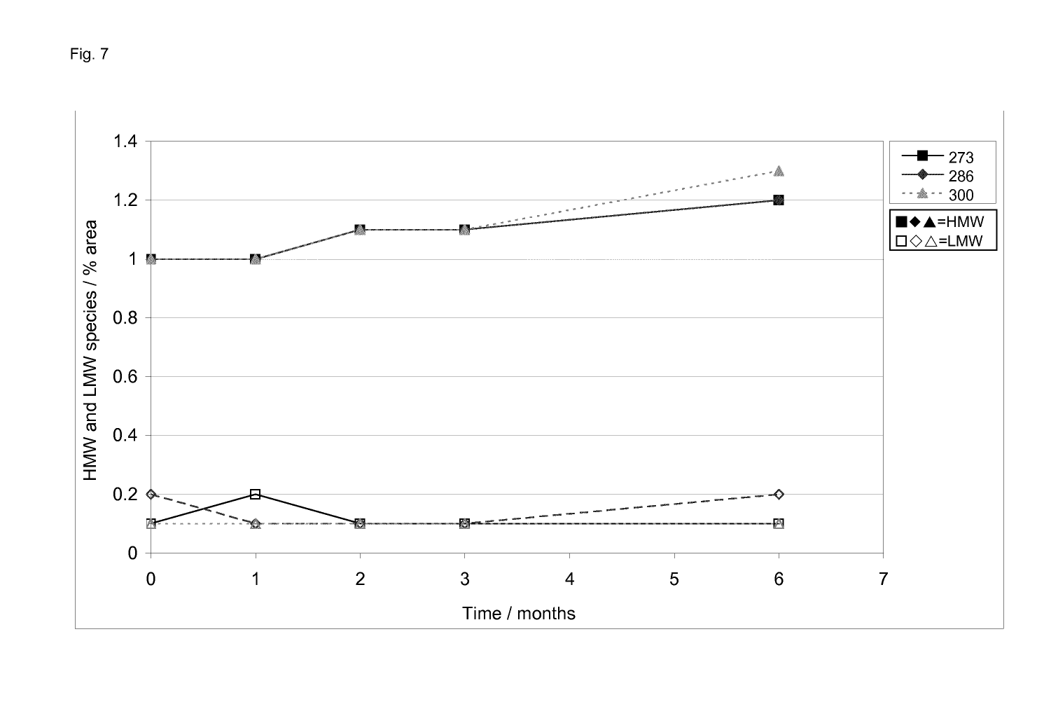

FIG. 8 shows the amount of HMW and LMW species per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 25° C. as a function of time. The level of HMW and LMW species per % area after 6 months for the liquid formulations filled into pre-filled syringes is also represented. Data were measured by size exclusion chromatography (SEC).

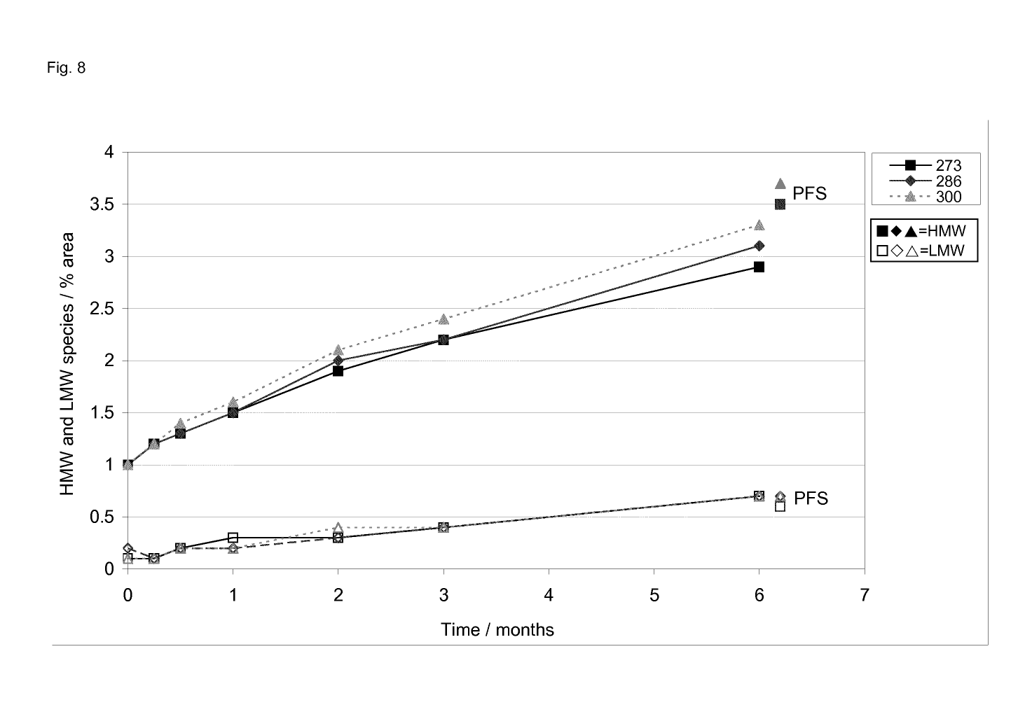

FIG. 9 shows the amount of HMW and LMW species per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 40° C. as a function of time. Data were measured by size exclusion chromatography (SEC).

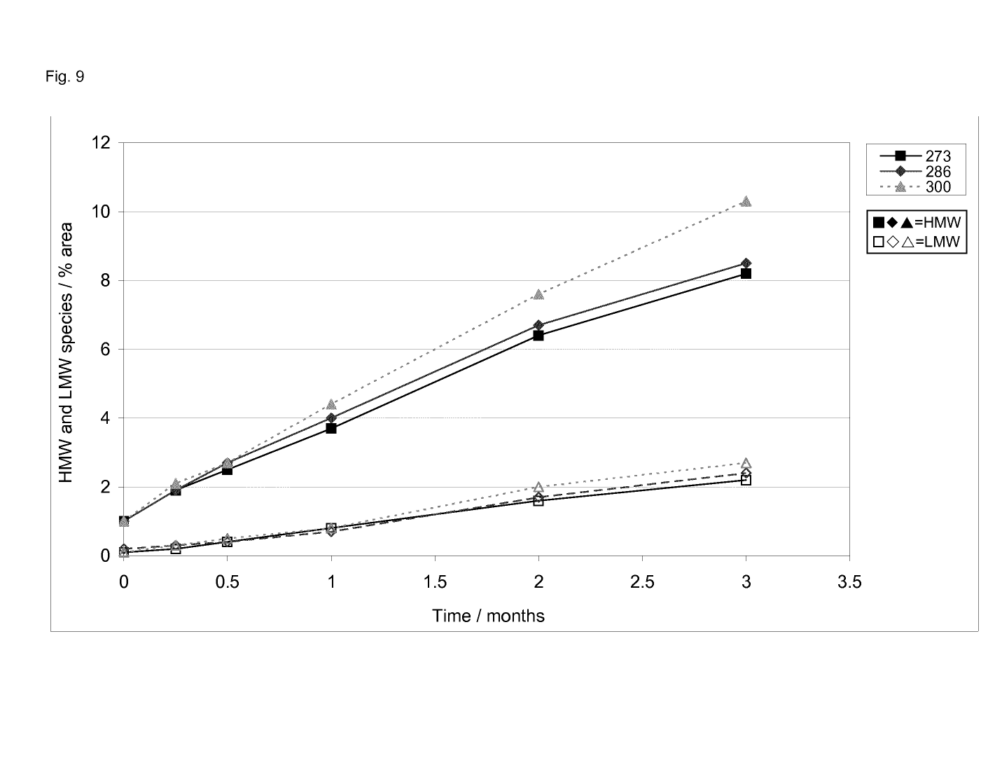

FIG. 10 shows the amount of acidic peak group (APG) per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively as a function of freeze thaw cycles. Data were measured by cation exchange chromatography (CEX).

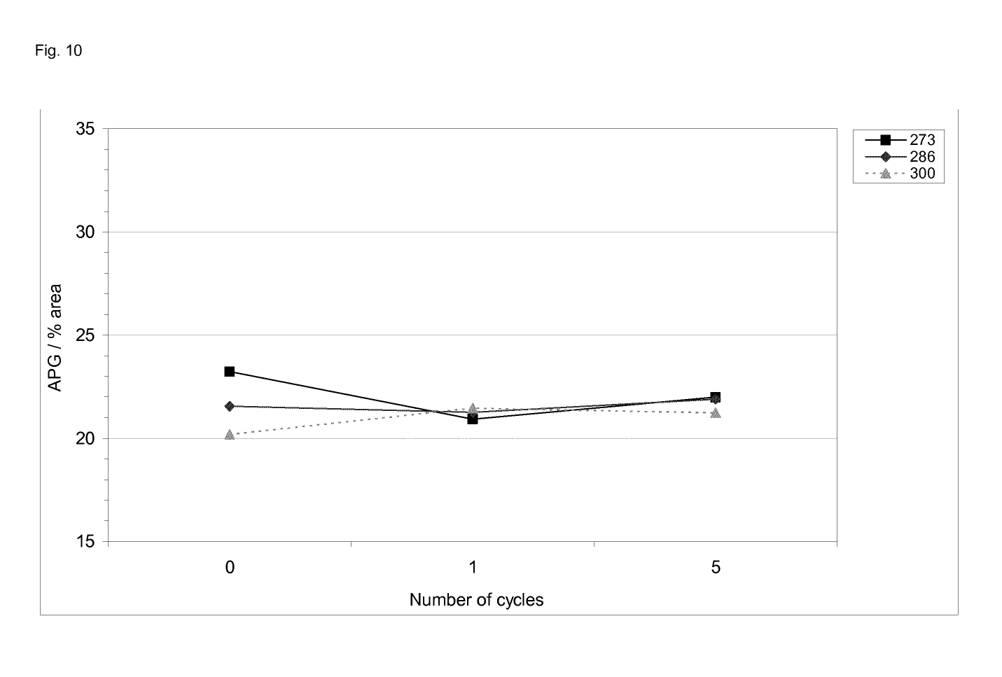

FIG. 11 shows the amount of acidic peak group (APG) per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 5° C. as a function of time. Data were measured by cation exchange chromatography (CEX).

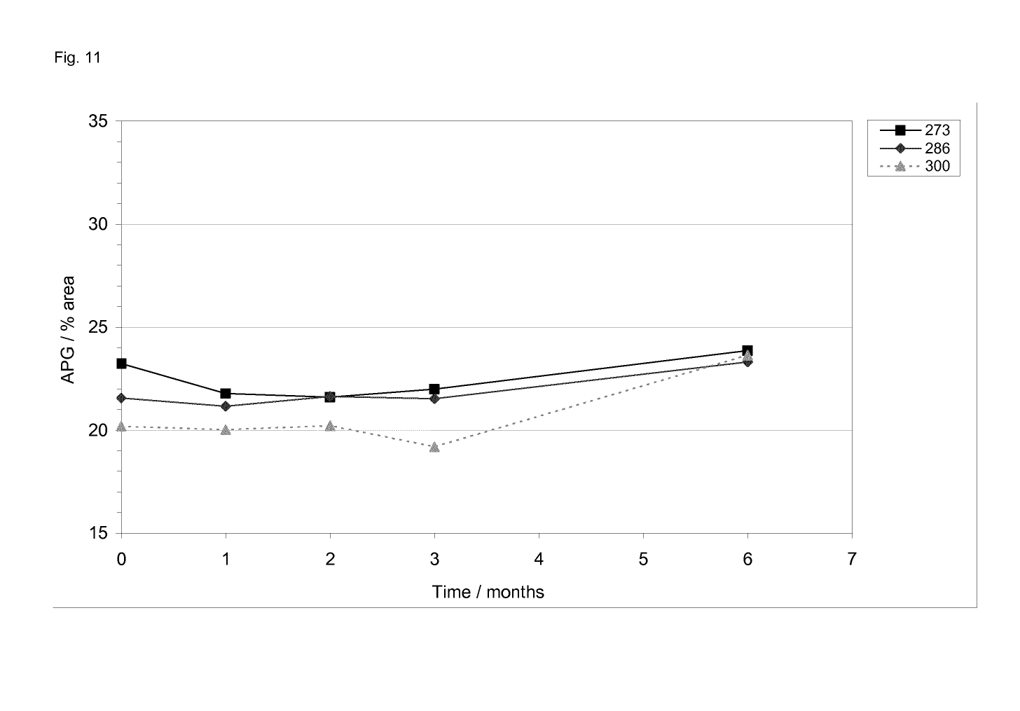

FIG. 12 shows the amount of acidic peak group (APG) per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 25° C. as a function of time. The level of APG per % area after 6 months for the liquid formulations filled into pre-filled syringes is also represented. Data were measured by cation exchange chromatography (CEX).

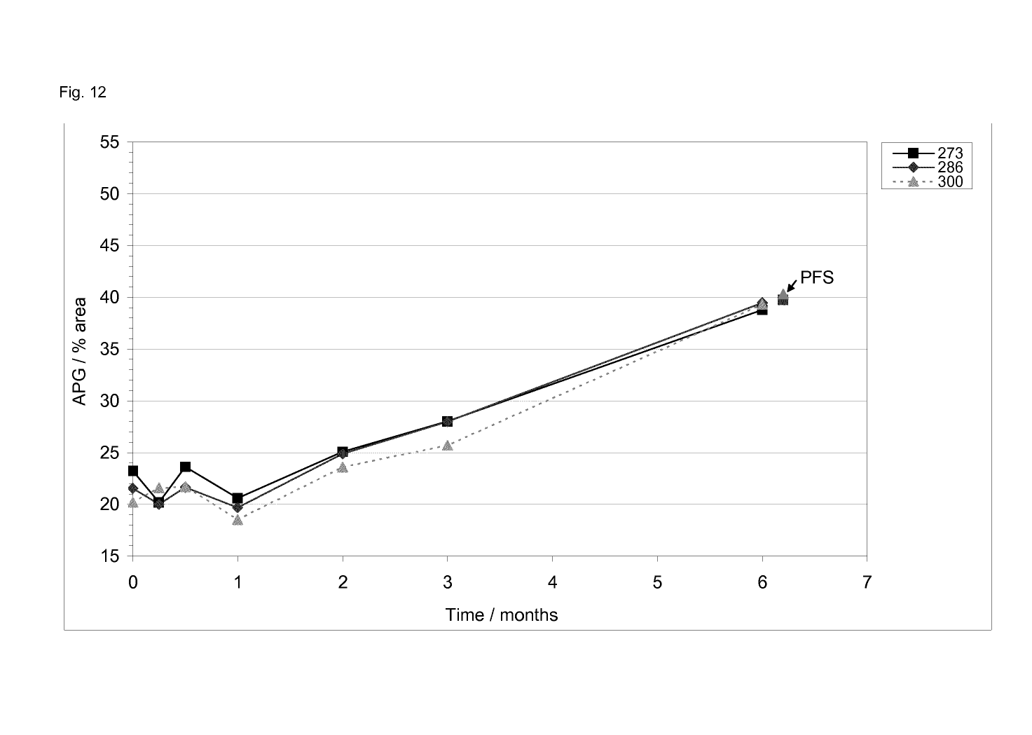

FIG. 13 shows the amount of acidic peak group (APG) per % area for the liquid formulation comprising 273 mg/mL epratuzumab, 40 mM sodium acetate, 220 mM glycine, 0.01% Polysorbate 80 at pH.5.0, 286 mg/mL epratuzumab, 55 mM sodium acetate, 220 mM glycine, 0.01% Polysorbate 80 at pH.5.0 and 300 mg/mL epratuzumab, 90 mM sodium acetate, 220 mM glycine, 0.01% Polysorbate 80 at pH.5.0, respectively stored at 40° C. as a function of time. Data were measured by cation exchange chromatography (CEX).

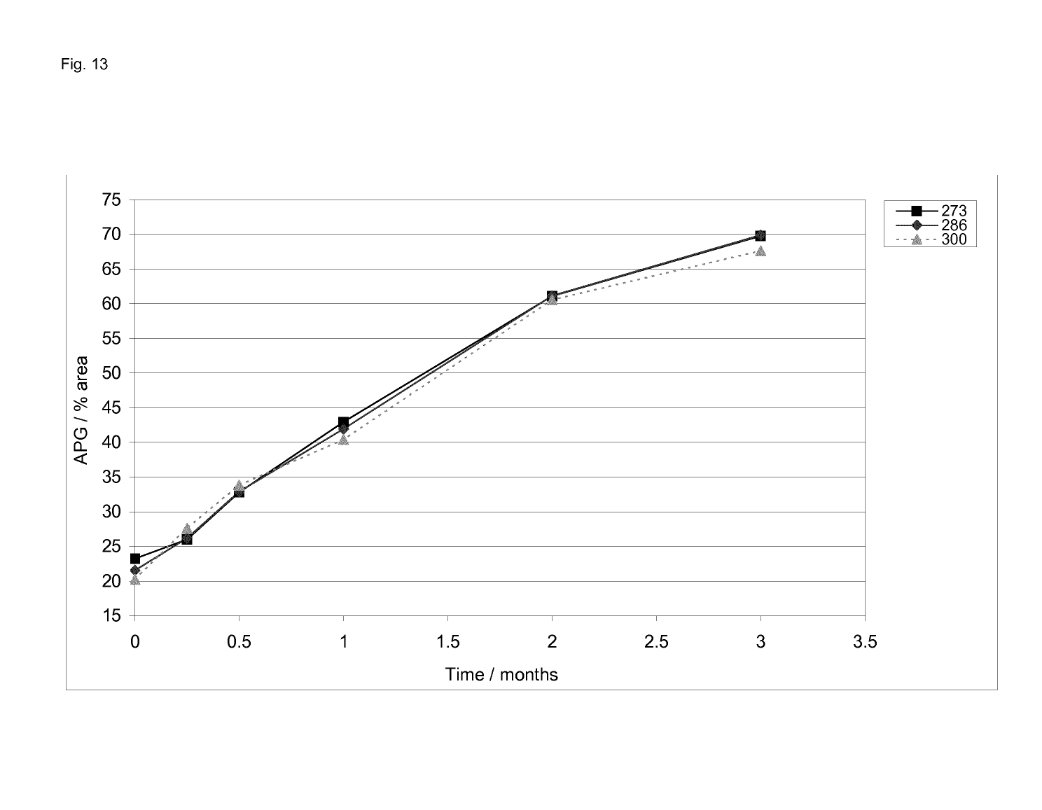

### DETAILED DESCRIPTION OF THE INVENTION

The present invention addresses the above-identified need by providing a novel stable liquid pharmaceutical formulation comprising a protein or an antibody having low viscosity and which is therefore suitable for subcutaneous administration to a mammalian, particularly human subject.

It is an object of the present invention to provide a liquid pharmaceutical formulation with reduced viscosity suitable for processing and administration comprising an antibody which is stable upon storage and transportation.

A stable formulation essentially retains the protein or antibody in solution essentially unaltered or minimally altered, preferably with no substantial decrease in bioactivity (e.g. equal or less than 5%, 10%, 20% or 30% decrease), after storage over time for example after one, two or three years of storage at about 5° C. In a stable formulation preferably the protein or antibody does not substantially aggregate or degrade with storage over time. Preferably the protein or antibody retains substantially its bioactivity with storage over time, for example after one, two or three years of storage at about 5° C.

In one embodiment according to the present invention a stable pharmaceutical formulation exhibits an increase of equal or less than 12%, preferably equal or less than 10%, more preferably equal or less than 5% and even more preferred equal or less than 3% in acid peak group (APG) species per % area in each case measured after three years of storage at about 5° C. Alternatively, a stable pharmaceutical formulation exhibits an increase of equal or less than 4%, preferably equal or less than 3.5%, more preferably equal or less than 2% and even more preferred equal or less than 1% in acid peak group (APG) species per % area in each case measured after one year of storage at about 5° C.

In another embodiment according to the present invention a stable pharmaceutical formulation exhibits an increase of equal or less than 10%, preferably equal or less than 5%, more preferably equal or less than 3% and even more preferred equal or less than 2% in high molecular weight (HMW) species per % area in each case measured after three years of storage at about 5° C. Alternatively, a stable pharmaceutical formulation exhibits an increase of equal or less than 3.5%, preferably equal or less than 2%, more preferably equal or less than 1% and even more preferred equal or less than 0.7% in high molecular weight (HMW) species per % area in each case measured after one year of storage at about 5° C.

In another embodiment according to the present invention a stable pharmaceutical formulation exhibits both the above limitations in the increase over time of APG species per % area and HMW species per % area.

“Acidic species” or “Acidic peak group (APG)” species as used herein refer to charge variants of an antibody or other protein which can result from a number of processes, including but not limited to deamidation, methionine oxidation, isomerization and hydrolysis. Charge variants are detected and quantified by ion exchange chromatography where they appear as distinct peaks reflecting a loss of positive charge or a gain in negative charge as compared with the parent peak of the unmodified antibody or protein. The amount of APG when measured by ion exchange chromatography such as cation exchange chromatography is commonly represented as APG/% area which refers to the ratio of the added area under all peaks in the chromatogram representing acidic species and the added area under all peaks in the same chromatogram.

“HMW species” or “LMW species” as used herein refer to higher molecular weight variants and lower molecular weight variants, respectively resulting from aggregation or degradation of a protein or antibody. HMW are also referred to as aggregates. The amount of HMW or LMW when measured by size exclusion chromatography (SEC) is commonly represented as HMW/% area or LMW/% area which refers to the ratio of the added area under all peaks in the SEC chromatogram representing HMW or LMW species, respectively and the added area under all peaks in the same chromatogram.

The term “antibody” or “antibodies” as used herein refers to monoclonal or polyclonal antibodies. The term “antibody” or “antibodies” as used herein includes but is not limited to recombinant antibodies that are generated by recombinant technologies as known in the art. “Antibody” or “antibodies” include antibodies' of any species, in particular of mammalian species, including antibodies having two essentially complete heavy and two essentially complete light chains, human antibodies of any isotype, including IgA₁, IgA₂, IgD, IgG1, IgG₂ₐ, IgG_{2b}, IgG₃, IgG₄ IgE and IgM and modified variants thereof, non-human primate antibodies, e.g. from chimpanzee, baboon, rhesus or cynomolgus monkey, rodent antibodies, e.g. from mouse, rat or rabbit; goat or horse antibodies, and camelid antibodies (e.g. from camels or llamas such as Nanobodies™) and derivatives thereof, or of bird species such as chicken antibodies or of fish species such as shark antibodies. The term “antibody” or “antibodies” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody performance. Humanization reduces the immunogenicity of non-human antibodies in humans, thus facilitating the application of antibodies to the treatment of human disease. Humanized antibodies and several different technologies to generate them are well known in the art. The term “antibody” or “antibodies” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies with specificity against a particular antigen upon immunization of the transgenic animal carrying the human germ-line immunoglobulin genes with said antigen. Technologies for producing such transgenic animals and technologies for isolating and producing the human antibodies from such transgenic animals are known in the art. Alternatively, in the transgenic animal; e.g. mouse, only the immunoglobulin genes coding for the variable regions of the mouse antibody are replaced with corresponding human variable immunoglobulin gene sequences. The mouse germline immunoglobulin genes coding for the antibody constant regions remain unchanged. In this way, the antibody effector functions in the immune system of the transgenic mouse and consequently the B cell development are essentially unchanged, which may lead to an improved antibody response upon antigenic challenge in vivo. Once the genes coding for a particular antibody of interest have been isolated from such transgenic animals the genes coding for the constant regions can be replaced with human constant region genes in order to obtain a fully human antibody. Other methods for obtaining human antibodies antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” or “antibodies” as used herein, also refers to an aglycosylated antibody.

The term “antibody” or “antibodies” as used herein not only refers to untruncated antibodies of any species, including from human (e.g. IgG) and other mammalian species, but also refers to an antibody fragment. A fragment of an antibody comprises at least one heavy or light chain immunoglobulin domain as known in the art and binds to one or more antigen(s). Examples of antibody fragments according to the invention include Fab, Fab′, F(ab′)₂, and Fv and scFv fragments; as well as diabodies, triabodies, tetrabodies, minibodies, domain antibodies, single-chain antibodies, bispecific, trispecific, tetraspecific or multispecific antibodies formed from antibody fragments or antibodies, including but not limited to Fab-Fv constructs. Antibody fragments as defined above are known in the art.

The term “monoclonal antibody” as used herein refers to a composition of a plurality of individual antibody molecules, wherein each individual antibody molecule is identical at least in the primary amino acid sequence of the heavy and light chains. For the most part, “monoclonal antibodies” are produced by a plurality of cells and are encoded in said cells by the identical combination of immunoglobulin genes. Generally “monoclonal antibodies” are produced by cells that harbor antibody genes, which are derived from a single ancestor B cell.

“Polyclonal antibody” or “polyclonal antibodies”, in contrast, refers to a composition of a plurality of individual antibody molecules, wherein the individual antibody molecules are not identical in the primary amino acid sequence of the heavy or light chains. For the most part, “polyclonal antibodies” bind to the same antigen but not necessarily to the same part of the antigen; i.e. antigenic determinant (epitope). Generally, “polyclonal antibodies” are produced by a plurality of cells and are encoded by at least two different combinations of antibody genes in said cells.

The antibody as disclosed herein is directed against an “antigen” of interest. Preferably, the antigen is a biologically important polypeptide and administration of the antibody to a mammal suffering from a disease or disorder can result in a therapeutic benefit in that mammal. However, antibodies directed against non-polypeptide antigens are also contemplated. Where the antigen is a polypeptide, it may be a transmembrane molecule (e.g. receptor) or ligand such as a growth factor or cytokine. Preferred molecular targets for antibodies encompassed by the present invention include CD polypeptides such as CD3, CD4, CD8, CD19, CD20, CD22, CD23, CD30, CD34, CD38, CD40, CD80, CD86, CD95 and CD154; members of the HER receptor family such as the EGF receptor, HER2, HER3 or HER4 receptor, cell adhesion molecules such as LFA-1, Mac1, p150,95, VLA-4, ICAM-1, VCAM and av/b3 integrin including either α or β subunits thereof (e.g. anti-CD11a, anti-CD18 or anti-CD11b antibodies), chemokines and cytokines or their receptors such as IL-1 α and β, IL-2, IL-6, the IL-6 receptor, IL-12, IL-13, IL-17 forms, IL-18, IL-21, IL-23, IL-25, IL-27, IFNγ, TNFα and TNFβ, growth factors such as VEGF, IgE, blood group antigens, flk2/flt3 receptor, obesity (OB) receptor, mpl receptor, CTLA-4, polypeptide C, G-CSF, G-CSF receptor, GM-CSF, GM-CSF receptor, M-CSF, M-CSF receptor, LINGO-1, BAFF, APRIL, OPG, OX40, OX40-L, β-amyloid and FcRn.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

The term “epratuzumab”, as used herein refers to the humanized antibody known in the art under the International Non-Proprietary Name (INN) epratuzumab. The light and heavy chain variable domain sequences of epratuzumab are depicted in SEQ ID NOs: 1 and 2, respectively.

The term “viscosity” as used herein, may be “kinematic viscosity” or “absolute viscosity.” Commonly, kinematic viscosity is expressed in centistokes (cSt). The SI unit of kinematic viscosity is mm 2/s, which is 1 cSt. Absolute viscosity is expressed in units of centipoise (cP). The SI unit of absolute viscosity is the millipascal second (mPa s), where 1 cP=1 mPa s.

The present invention provides a stable liquid pharmaceutical formulation comprising a protein or an antibody as active ingredient and acetate.

In a first embodiment of the invention the liquid pharmaceutical formulation comprises a protein or an antibody at a concentration of 200 to 400 mg/ml, 220 to 380 mg/ml, 250 to 350 mg/ml, 270 to 310 mg, 280 to 300 mg/ml, 273 mg/ml or 286 mg/ml or 300 mg/ml.

In the second embodiment the liquid pharmaceutical formulation of the first embodiment of the invention comprises acetate, preferably, sodium acetate, at a concentration of 20 to 150 mM, 30 to 120 mM, 40 to 90 mM, 50 to 75 mM, equal or at least 40 mM, equal or at least 55 mM or equal or at least 90 mM.

In the third embodiment the liquid pharmaceutical formulation of the first or second embodiment of the invention comprises glycine at a concentration of 100 to 500 mM, 150 to 500 mM, 100 to 450 mM, 150 to 450 mM, 150 to 350 mM, 220 to 420 mM or 250 to 350 mM.

In the fourth embodiment the liquid pharmaceutical formulation of the first, second or third embodiment of the invention the osmolality of the pharmaceutical formulation is 250 to 650 mOsm/kg, 250 to 550 mOsm/kg, 250 to 500 mOsm/kg, 250 to 450 mOsm/kg, 275 to 425 mOsm/kg, 275 to 410 mOsm/kg, 300 to 410 mOsm/kg or 275 to 300 mOsm/kg.

In the fifth embodiment the liquid pharmaceutical formulation of the first, second, third or fourth embodiment of the invention has a viscosity of equal or less than 110 mPa s, equal or less than 100 mPa s, equal or less than 90 mPa s, equal or less than 80 mPa s, equal or less than 70 mPa s, 50 to 110 mPa s, 50 to 100 mPa s, 60 to 100 mPa s or 60 to 90 mPa s.

In the sixth embodiment the liquid pharmaceutical formulation of the first, second, third, fourth or fifth embodiment of the invention has a pH of 4.0 to 7.0, 4.5 to 6.5, 5.0 to 6.0 or 5.0.

In the seventh embodiment the liquid pharmaceutical formulation of the first, second, third, fourth, fifth or sixth embodiment of the invention comprises NaCl at a concentration of 0 to 100 mM, 10 to 90 mM, 20 to 80 mM, 30 to 70 mM, 40 to 60 mM or 50 mM.

In the eighth embodiment the liquid pharmaceutical formulation of the first, second, third, fourth, fifth, sixth or seventh embodiment of the invention comprises a surfactant, preferably Polysorbate 80.

In the ninth embodiment the liquid pharmaceutical formulation of the first, second, third, fourth, fifth, sixth, seventh or eighth embodiment of the invention comprises Polysorbate 80 at a concentration of 0.001 to 0.03% w/v, 0.005 to 0.025% w/v or 0.01 to 0.02% w/v.

In the tenth embodiment the liquid pharmaceutical formulation of the first, second, third, fourth, fifth, sixth, seventh, eighth or ninth embodiment of the invention does not comprise a divalent cation.

In the eleventh embodiment the liquid pharmaceutical formulation of the first, second, third, fourth, fifth, sixth, seventh, eighth, ninth or tenth embodiment of the invention does not comprise MgCl₂ or CaCl₂.

In the twelfth embodiment the liquid pharmaceutical formulation of the first, second, third, fourth, fifth, sixth, seventh, eighth, ninth, tenth or eleventh embodiment of the invention comprises an antibody, preferably an untruncated antibody or an antibody fragment or derivative.

In the thirteenth embodiment the liquid pharmaceutical formulation of the first, second, third, fourth, fifth, sixth, seventh, eighth, ninth, tenth, eleventh or twelfth embodiment of the invention the antibody is epratuzumab.

The fourteenth embodiment of the invention is a container, preferably a syringe or another injection device, such as an autoinjector or a cartridge or other container for use with an injection device, containing the liquid pharmaceutical formulation of the first, second, third, fourth, fifth, sixth, seventh, eighth, ninth, tenth, eleventh, twelfth or thirteenth embodiment of the invention. A useful container is a vial made of glass or another material, or a bag made of synthetic material.

The sixteenth embodiment of the invention is a kit comprising the container of the fourteenth embodiment of the invention and instructions for use.

The seventeenth embodiment of the invention is a method for reducing the viscosity of a liquid pharmaceutical formulation containing a protein or an antibody, the method comprising providing the liquid pharmaceutical formulation, preferably at a concentration of 200 to 400 mg/ml, 220 to 380 mg/ml, 250 to 350 mg/ml, 270 to 310 mg, 280 to 300 mg/ml, 273 mg/ml or 286 mg/ml or 300 mg/ml, and adding acetate, preferably sodium acetate, to a final concentration of 20 to 150 mM, 30 to 120 mM, 40 to 90 mM, 50 to 75 mM, equal or at least 40 mM, equal or at least 55 mM or equal or at least 90 mM, wherein the viscosity of the liquid pharmaceutical formulation is reduced as compared to the same liquid pharmaceutical formulation without acetate.

In a further preferred embodiment of the invention the liquid pharmaceutical formulation of any of the embodiments one to seventeen exhibits an opalescence which is ≧Reference Standard II and ≦Reference Standard III as defined in the European Pharmacopeia, section 2.2.1. (Clarity and degree of opalescence of liquids) and corresponds to ≧6 NTU and ≦18 NTU (nephelometric turbidity units). The opalescence may be determined after filling into a container, or after one year of storage at about 5° C., or after two years of storage at about 5° C., or after three years of storage at about 5° C.

In a further embodiment the liquid pharmaceutical formulation of any of the embodiments disclosed herein comprises a surfactant selected from the group consisting of poloxamer (e.g. poloxamer 188), Triton, sodium dodecyl sulfate (SDS), sodium laurel sulfate, sodium octyl glycoside, lauryl-, myristyl-, linoleyl-, or stearyl-sulfobetaine, lauryl-, myristyl-, linoleyl- or stearyl-sarcosine, linoleyl-, myristyl-, or cetyl-betaine, lauroamidopropyl-, cocamidopropyl-, linoleamidopropyl-, myristamidopropyl-, palmidopropyl-, or isostearamidopropyl-betaine, myristamidopropyl-, palmidopropyl-, or isostearamidopropyl-dimethylamine, sodium methyl cocoyl-, or disodium methyl oleyl-taurate, polyethyl glycol, polypropyl glycol, and copolymers of ethylene and propylene glycol.

In a further embodiment the liquid pharmaceutical formulation of any of the embodiments disclosed herein further comprises a stabilizer. Stabilizers according to the present invention include sucrose, trehalose, mannitol, sorbitol and arginine hydrochloride.

Optionally, preservatives may be used in the liquid pharmaceutical formulation of the invention. Suitable preservatives for use in the liquid pharmaceutical formulation of the invention include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

Other pharmaceutically acceptable carriers, excipients or stabilizers such as those described in Remington's Science and Practice of Pharmacy 21^{st} edition, (2005) or Loyd V. Allen, Art, Science and Technology of Pharmaceutical Compounding, 3^{rd} edition (2008), ISBN 1582121109 may be included in the liquid pharmaceutical formulation of the invention provided that they do not adversely affect the desired characteristics of the formulation. Acceptable carriers, excipients or stabilizers are nontoxic to recipients at the dosages and concentrations employed and include additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counter-ions such as sodium.

In a further embodiment the invention provides a method for treating a mammal, particularly human subject comprising administering a therapeutically effective amount of the liquid pharmaceutical formulation of any of the embodiments disclosed herein to a mammal, particularly human subject wherein the mammal, particularly human subject has a disorder that may be ameliorated through treatment with the liquid pharmaceutical formulation.

In a further embodiment the invention provides a method for treating a mammal, particularly human subject comprising administering a therapeutically effective amount of the liquid pharmaceutical formulation of any of the embodiments disclosed herein comprising epratuzumab as an active ingredient to a mammal, particularly human subject wherein the mammal, particularly human subject has a disorder that may be ameliorated through treatment with epratuzumab, whereby the disorder is an autoimmune or inflammatory disease, particularly an autoimmune or inflammatory disease in which B-cells are implicated in the pathophysiology and/or the symptoms of disease. Such autoimmune diseases and inflammatory disease may also be referred to as B-cell mediated autoimmune diseases or inflammatory disease: B-cells have been implicated in playing a role in the pathophysiology of a variety of autoimmune or inflammatory diseases. For example, autoimmune diseases and inflammatory disease include but are not limited to rheumatoid arthritis, systemic lupus erythematosus, Sjögren's syndrome, ANCA-associated vasculitis, antiphospholipid syndrome, idiopathic thrombocytopaenia, autoimmune haemolytic anaemia, Guillian-Barré syndrome, chronic immune polyneuropathy, autoimmune thryoiditis, type I diabetes, Addison's disease, membranous glomerulonephropathy, Goodpasture's disease, autoimmune gastritis, pernicious anaemia, pemiphigus vulgarus, primary biliary cirrhosis, dermatomyositis-polymyositis, myasthenia gravis, celiac disease, immunoglobulin A nephropathy, Henoch-Schönlein purpura, chronic graft rejection, atopic dermatitis, asthma, allergy, systemic sclerosis, multiple sclerosis, Lyme neuroborreliosis, ulcerative colitis, interstitial lung disease.

In a further embodiment of the invention the liquid pharmaceutical formulation comprising epratuzumab as an active ingredient further comprises or is administered in combination with (at the same time point of at a different time point) one or more additional therapeutic agents, such as, for example, a corticosteroid, a non-steroidal anti-inflammatory drug (NSAIDs), chloroquine, hydroxycloroquine, methotrexate, leflunomide, azathioprine, mycophenolate mofetil, cyclophosphamide, chlorambucil, and cyclosporine, mycophenolate mofetil, a CD20 antagonist, such as rituximab, ocrelizumab, veltuzumab or ofatumumab, abatacept, a TNF antagonist, such as etanercept, tacrolimus, sirolimus, dehydroepiandrosterone, lenalidomide, an IL-6 or IL-6 receptor antagonist, such as olokizumab, tocilizumab, AMG811, CNTO136, BMS-945429 (formerly ALD518), sarilumab, sirukumab, a CD40 or CD40-L antagonist, such as anti-CD40 or anti-CD40L antibodies, an OX40 or OX40-L antagonist, rontalizumab, rigerimod, sifalimumab, AGS-009, atacicept, laquinimod, abetimus sodium and/or belimumab.

In a further embodiment the invention provides a method for treating a mammal, particularly human subject comprising administering a therapeutically effective amount of the liquid pharmaceutical formulation of any of the embodiments disclosed herein comprising epratuzumab as an active ingredient to a mammal, particularly human subject wherein the mammal, particularly human subject has a disorder that may be ameliorated through treatment with epratuzumab, whereby the disorder is cancer such as for example, leukemia and non-Hodgkin lymphoma, acute lymphoblastic leukemia, acute myeloid leukemia, adult, acute myeloid leukemia, adrenocortical carcinoma, astrocytoma, basal cell carcinoma, bile duct cancer, bladder cancer, bone cancer, such as osteosarcoma and malignant fibrous histiocytoma, glioma, ependymoma, medulloblastoma, breast cancer, bronchial adenomas, cervical cancer, chronic lymphocytic leukemia, chronic myelogenous leukemia, colon cancer, colorectal cancer, endometrial cancer, esophageal cancer, Ewing's family of tumors, extracranial germ cell tumor, extragonadal germ cell tumor, extrahepatic bile duct cancer, retinoblastoma, gallbladder cancer, gastric (stomach) cancer, gastrointestinal carcinoid tumor, gastrointestinal stromal tumor (GIST), germ cell tumor, gestational trophoblastic tumor, hairy cell leukemia, head and neck cancer, hepatocellular (liver) cancer, lymphoma, such as Hodgkin's lymphoma, Burkitt's lymphoma, cutaneous T-cell lymphoma, such as mycosis fungoides and Sezary syndrome, hypopharyngeal cancer, melanoma, such as intraocular melanoma, Kaposi's sarcoma, kidney (renal cell) cancer, laryngeal cancer, lip and oral cavity cancer, lung cancer, such as non-small cell lung cancer or small cell lung cancer, Waldenstrom's macroglobulinemia, Merkel cell carcinoma, mesothelioma, mouth cancer, multiple myeloma, myelodysplastic syndromes, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary tumor, plasma cell neoplasm, pleuropulmonary blastoma, prostate cancer, rectal cancer, rhabdomyosarcoma, salivary gland cancer, sarcoma, testicular cancer, throat cancer, thymoma, thyroid cancer, urethral cancer, or Wilms' tumor.

In a further embodiment of the invention the liquid pharmaceutical formulation comprising epratuzumab as an active ingredient further comprises or is administered in combination with (at the same time point of at a different time point) one or more additional therapeutic agents, such as, for example, another, a compound that inhibitis the activity or activation of the EGF-R pathway (e.g. cetuximab, panatimumab, zalutumumab, nimotuzumab, matuzumab, trastuzumab, pertuzumab, gefitinib, erlotinib, lapatinib, EKB-569, HKI-272, CI-1033, vandetanib or BIBW2992); a tyrosine kinase inhibitor (e.g. sorafenib, sutinib, imatinib, dasatinib, valatinib, sonitinib, ofimatinib, AEE788); an anti-angiogenic agent, such as thalidomide, lenalidomide, a VEGF or a VEGF-R antagonist (e.g. VEGF-RI, VEGF-R2) (e.g. bevacizumab, VEGF-trap, pegaptanib, vandetanib, vatalanib, cediranib, ranibizumab, aflibercept, enzastaurin, cediranib, SU-4984, SU-5402, PD-173074), an FGF antagonist (e.g. FGFI, FGF2, FGF-3, FGF4, FGF5, FGFG, FGF7, FGF84, FGF9, FGFIO, FGFII, FGF12, FGF13, FGF14, FGF16, FGF17, FGF18, FGF19, FGF20, FGF21, FGF22, FGF23) or FGF-R (e.g. FGF-R1, FGF-R2, FGF-R3, FGF-R4) antagonist; an IL-8 antagonist (e.g. an anti-IL-8 antibody such as MDX018/HuMax-Inflam); procarbazine; mechlorethamine; cyclophosphamide; camptothecin; carmustine; ifosfamide; melphalan; chlorambucil; busulfan; dactinomycin; daunorubicin; doxorubicin; bleomycin; plicomycin; mitomycin; tamoxifen; raloxifene; an estrogen receptor binding agent; paclitaxel; gemcitabine; navelbine; a farnesyltransferase inhibitor (e.g. lonafarnib, tipifarnib); an inhibitor of mTOR (mammalian target of rapamycin) (e.g. sirolimus; temirolimus; everolimus, deforolimus); an integrin inhibitor (e.g. cilengitide, the monoclonal antibodies CNTO95 and etaracizumab all blocking the αᵥβ₃ integrin, or the monoclonal antibody volociximab blocking the α₅β₁ integrin); an inhibitor of the poliovirus receptor (PVR/CD155/Necl-5); an inhibitor of the cytoskeleton (e.g. taxol, eleutherobin, colcimid, nocodazole, discodermolide, epithilone, ixabepilone, epothilone B, cemadotin, dolastin, rhizoxin, combretastatin, maytansine, monomethylauristatin E, or other auristatin derivatives, extramustine, cytochalasin, vincristin or colchicin); an inhibitor of protein disulfide isomerase; an MMP inhibitor; a c-SRC inhibitor (e.g. AP22408, AZD0530, AZM475271, BMS-354825, CGP77675, 17-AAG, PP2, SKI-606, SU6656, anilinoquinazolines, PD173952, PD173955, terphenylquinone or UCSI 5A); transplatinum; 5-fluorouracil; capecitabine; tegafur-uracil; bortezomib; gemcitabine; methotrexate; temozolomide; nitrosourea; cisplatin; carboplatin; satraplatin; vincristin; vinblastin; vindesine; bendamustine; ecteinascidin-743; netropsin; podophyllotoxin; etoposide; teniposide; lexitropsin; enediyne; duocarmycine; irinotecan; oxiplatin; edotecarin or an inhibitor of topoisomerase I or II (e.g. topotecan).

The liquid pharmaceutical formulation of the invention is suitably administered to the patient at one time or over a series of treatments and may be administered to the patient at any time from diagnosis onwards; it may be administered as the sole treatment or in conjunction with other drugs or therapies useful in treating the conditions as described herein before.

For the treatment of the above diseases, the appropriate dosage will vary depending upon, for example, the particular antibody to be employed, the subject treated, the mode of administration and the nature and severity of the condition being treated. Preferred dosage regimen for treating autoimmune diseases and inflammatory disease with the liquid pharmaceutical formulation of the present invention comprising epratuzumab as an active ingredient are disclosed in WO 2011/032633 and comprise, for example, the administration of epratuzumab in an amount of 400 to 800 mg, preferably, 600 mg, once every week for 4 consecutive weeks in a treatment cycle of 12 weeks or in an amount of 1000 to 1400 mg, preferably 1200 mg, once every other week for 4 consecutive weeks in a treatment cycle of 12 weeks.

The pharmaceutical formulation according any of the embodiments of the invention is administered preferably by the subcutaneous injection route. The pharmaceutical formulation according any of the embodiments of the invention may also be administered by intramuscular or intravenous injection route. The pharmaceutical formulation may be injected using a syringe or an injection device such as an autoinjector. A preferred syringe for administration of the pharmaceutical formulation according any of the embodiments of the invention has a user-friendly design that allows subjects to more easily administer the pharmaceutical formulation of the invention, particularly subjects with, for example, compromised dexterity or joint strength. An example of such syringe is disclosed in WO 2009/090499. A preferred injection device for administering the pharmaceutical formulation according any of the embodiments of the invention is a reusable housing, a syringe assembly that is slidably mounted on the housing, a needle, a fluid container, an autoinjector actuator for urging the syringe assembly with respect to the housing from a storage position to a launch position, and an improved cap that releasably engages with the housing, such as for example disclosed in WO 2010/007395.

As used herein, “a” or an may mean one or more. As used herein, when used in conjunction with the word “comprising”, the words “a” or an may mean one or more than one. As used herein “including” may mean including without limitation.

The use of the term or as used herein mean “and/or” unless explicitly indicated to refer to alternatives only or the alternatives are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

### EXAMPLES

### Example 1

Different pharmaceutical formulations of epratuzumab at a concentration of 300 mg/mL were prepared. The concentration of sodium acetate (NaOAc) and sodium chloride (NaCl) was varied in order to investigate the effect of these salts on the viscosity of the formulation.

Materials were concentrated and buffer exchanged using viva flow cassettes and spin tubes. Formulation concentrations were confirmed using a Solo-VPE high concentration variable path length UV/Vis spectrophotometer (C Technologies, Inc., Bridgewater, N.J., USA) and viscosity determined using a RVDV-II viscometer (Brookfield Engineering Laboratories, Inc., Massachusetts, USA).

Ten separate formulations were generated (from an initial protein stock of 6 mg/mL) from a combination of replicates and Design of Experiments (DoE) to investigate the relationships between antibody, glycine, NaCl, NaOAc, and Polysorbate 80 (PS80) concentration all at pH 5.0.

TABLE 1
Table of formulation based on 200 mg/mL epratuzumab,
220 mM, pH 5.0 based formulation

| Formulation | NaOAc (mM) | Polysorbate 80 (%) | NaCl | Viscosity/cP |
|---|---|---|---|---|
| 1 | 30 | 0.01 | 0 | 190.75 |
| 2 | 40 | 0 | 0 | 181.99 |
| 3 | 40 | 0.01 | 0 | 140.57 |
| 4 | 40 | 0.03 | 0 | 139.76 |
| 5 | 60 | 0.01 | 0 | 105.08 |
| 6 | 60 | 0 | 0 | 103.03 |
| 7 | 80 | 0 | 0 | 91.56 |
| 8 | 30 | 0 | 0 | 164.76 |
| 9 | 60 | 0.01 | 50 | 51.80 |
| 10 | 60 | 0.01 | 100 | 48.29 |

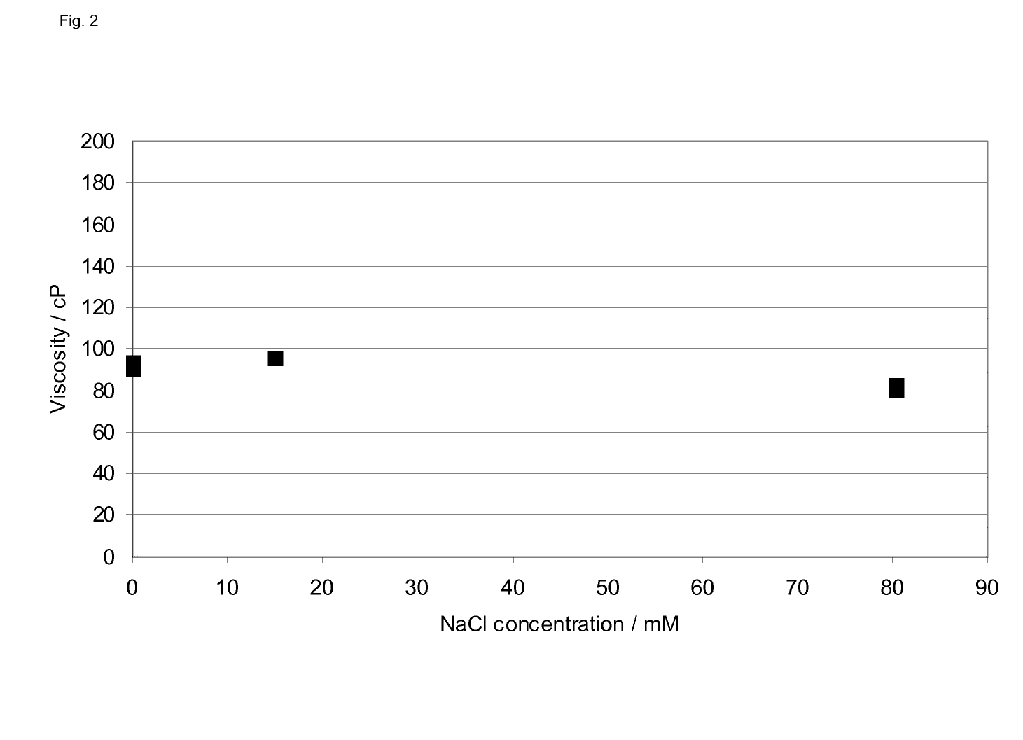

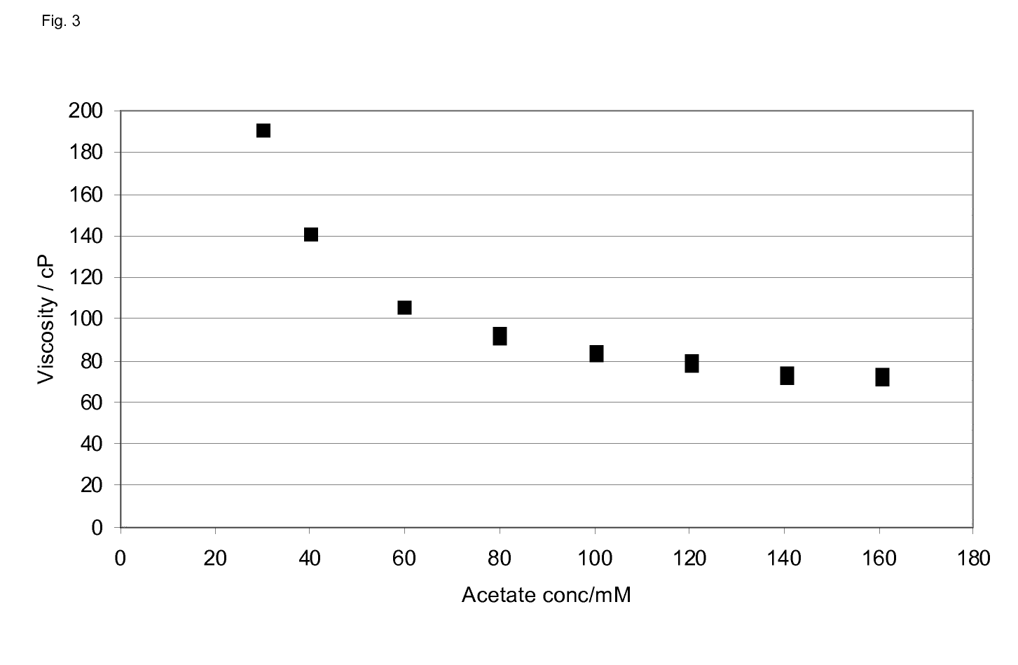

Based on the results of the experiments it was concluded that surprisingly NaOAc has a greater effect on reducing the viscosity of high concentration formulations of antibodies than NaCl. FIG. 2 shows the reduction in the viscosity of the 273 mg/mL, 220 mM glycine, 60 mM NaOAc formulation observed with varying NaCl concentrations (0, 50, and 100 mM), with 100 mM NaCl the viscosity reduced from 61.7 cP (0 mM) to 48.3 cP (100 mM) a 21.7% reduction. FIG. 3 shows the reduction in viscosity observed due to an increase of NaOAc. Reduction in viscosity of 300 mg/mL material with 220 mM glycine and NaOAc between 30-60 mM, with a reduction in viscosity from 164.8 cP (30 mM) to 103.03 cP (60 mM) a 37.3% reduction with a 30 mM increase.

### Example 2

Further experiments were performed in order to investigate the effect of NaOAc on the viscosity of high concentration antibody formulations.

Materials were concentrated and buffer exchanged using viva flow cassettes and spin tubes. Formulation concentrations were confirmed using a Solo-VPE high concentration variable path length UV/Vis spectrophotometer (C Technologies, Inc., Bridgewater, N.J., USA) and viscosity determined using a RVDV-II viscometer (Brookfield Engineering Laboratories, Inc., Massachusetts, USA). Osmolality of the pharmaceutical formulations was determined using a vapor pressure osmometer (Vapro® 5520, Wescor, Inc., Utah, USA).

Formulations in Table 2 (30 formulations) and Table 3 (12 formulation in duplicate/triplicate) were prepared and analyzed in triplicate, where there was insufficient material, formulations were tested in duplicate.

TABLE 2
DoE Formulation Generation for Response Surface Map (RSM)

| | NaOAc | Glycine | | NaCl | | epratuzumab |
|---|---|---|---|---|---|---|
| Formulation | (mM) | (mM) | PS80 (%) | (mM) | pH | (mg/mL) |
| 1 | 40 | 220 | 0.015 | 50 | 5 | 270 |
| 2 | 40 | 220 | 0.03 | 0 | 5 | 290 |
| 3 | 40 | 220 | 0 | 0 | 5 | 290 |
| 4 | 40 | 220 | 0.015 | 0 | 5 | 270 |
| 5 | 60 | 220 | 0.015 | 50 | 5 | 270 |
| 6 | 40 | 220 | 0 | 100 | 5 | 250 |
| 7 | 40 | 220 | 0.015 | 100 | 5 | 270 |
| 8 | 40 | 220 | 0.015 | 50 | 5 | 270 |
| 9 | 40 | 220 | 0 | 50 | 5 | 270 |
| 10 | 40 | 220 | 0.015 | 50 | 5 | 250 |
| 11 | 40 | 220 | 0 | 100 | 5 | 290 |
| 12 | 40 | 220 | 0.03 | 0 | 5 | 250 |
| 13 | 40 | 220 | 0.015 | 50 | 5 | 290 |
| 14 | 50 | 220 | 0.015 | 50 | 5 | 270 |
| 15 | 40 | 220 | 0.015 | 50 | 5 | 270 |
| 16 | 40 | 220 | 0.03 | 50 | 5 | 270 |
| 17 | 40 | 220 | 0 | 0 | 5 | 250 |
| 18 | 40 | 220 | 0.03 | 100 | 5 | 250 |
| 19 | 40 | 220 | 0.03 | 100 | 5 | 290 |
| 20 | 40 | 220 | 0.015 | 50 | 5 | 270 |
| 21 | 40 | 220 | 0 | 0 | 5 | 290 |
| 22 | 40 | 220 | 0 | 100 | 5 | 250 |
| 23 | 40 | 220 | 0.015 | 0 | 5 | 290 |
| 24 | 30 | 220 | 0.015 | 50 | 5 | 270 |
| 25 | 60 | 220 | 0.015 | 35 | 5 | 300 |
| 26 | 40 | 220 | 0.015 | 55 | 5 | 300 |
| 27 | 60 | 220 | 0.015 | 85 | 5 | 300 |
| 28 | 80 | 220 | 0.015 | 15 | 5 | 300 |
| 29 | 60 | 220 | 0.015 | 35 | 5 | 290 |
| 30 | 80 | 220 | 0.015 | 15 | 5 | 290 |

TABLE 3
DoE Formulation Generation for Drug
Substance (DS) manufacturing

| | NaOAc | Glycine | | NaCl | | epratuzumab |
|---|---|---|---|---|---|---|
| Formulation | (mM) | (mM) | PS80 (%) | (mM) | pH | (mg/mL) |
| 1 | 80 | 220 | 0.01 | 0 | 5 | 300 |
| 2 | 100 | 220 | 0.01 | 0 | 5 | 300 |
| 3 | 120 | 220 | 0.01 | 0 | 5 | 300 |
| 4 | 140 | 220 | 0.01 | 0 | 5 | 300 |
| 5 | 160 | 220 | 0.01 | 0 | 5 | 300 |
| 6 | 80 | 220 | 0.01 | 0 | 5 | 300 |
| 7 | 86 | 242 | 0.01 | 0 | 5 | 330 |
| 8 | 130 | 242 | 0.01 | 0 | 5 | 330 |
| 9 | 174 | 242 | 0.01 | 0 | 5 | 330 |
| 10 | 89 | 253 | 0.01 | 0 | 5 | 345 |
| 11 | 135 | 253 | 0.01 | 0 | 5 | 345 |
| 12 | 181 | 253 | 0.01 | 0 | 5 | 345 |

TABLE 4
Results Summary DoE Formulation Generation

| | | Osmolarity |
|---|---|---|
| Formulation | Viscosity (cP) | (mOsmol/L) |
| 1 | 52.80 | ¹ |
| 2 | 106.93 | ¹ |
| 3 | 107.81 | ¹ |
| 4 | 65.47 | 307 |
| 5 | 48.25 | 458 |
| 6 | 31.78 | 533 |
| 7 | 48.29 | 548 |
| 8 | 52.02 | 472 |
| 9 | 52.63 | 438 |
| 10 | 33.43 | 400 |
| 11 | 84.13 | ¹ |
| 12 | 39.68 | 277 |
| 13 | 88.18 | ² |
| 14 | 50.42 | ¹ |
| 15 | 52.53 | 460 |
| 16 | 52.01 | 428 |
| 17 | 40.12 | 272 |
| 18 | 30.84 | 506 |
| 19 | 83.17 | ¹ |
| 20 | 52.40 | ¹ |
| 21 | 108.79 | ¹ |
| 22 | 31.28 | 505 |
| 23 | 104.93 | ¹ |
| 24 | 55.85 | ¹ |
| 25 | 112.23 | ¹ |
| 26 | 113.97 | ¹ |
| 27 | 96.47 | ¹ |
| 28 | 95.52 | ¹ |
| 29 | 82.62 | ¹ |
| 30 | 70.61 | ² |

¹ sample too viscous,
² insufficient material

TABLE 5
Results Summary DoE Formulation Generation Continued

| | Viscosity (cP) | | | | |
|---|---|---|---|---|---|
| Formulation | Replicate 1 | Replicate 2 | Replicate 3 | Mean | ± |
| 1 | 93.52 | 90.86 | 90.91 | 91.76 | 1.52 |
| 2 | 84.58 | 82.88 | 83.32 | 83.59 | 0.88 |
| 3 | 79.57 | 79.88 | 77.74 | 79.06 | 1.16 |
| 4 | 74.21 | 72.72 | 71.63 | 72.85 | 1.30 |
| 5 | 73.42 | 72.33 | 71.55 | 72.43 | 0.94 |
| 6 | 82.58 | 82.53 | 80.09 | 81.73 | 1.42 |
| 7 | 250.35 | 236.31 | 235.96 | 240.87 | 8.21 |
| 8 | 199.08 | 197.60 | 197.16 | 197.95 | 1.01 |
| 9 | 177.28 | 181.20 | ¹ | 179.24 | 2.77 |
| 10 | 359.09 | 352.46 | 341.65 | 351.07 | 8.80 |
| 11 | 293.95 | 272.33 | ¹ | 283.14 | 15.29 |
| 12 | 269.01 | 272.33 | ¹ | 270.67 | 2.35 |

¹ insufficient material

### REFERENCE LIST

Andya, J. D., Hsu, C. C., & Shire, S. J. (2003). Mechanisms of aggregate formation and carbohydrate excipient stabilization of lyophilized humanized monoclonal antibody formulations. AAPS. PharmSci. 5, E10.
Galush, W. J., Le, L. N., & Moore, J. M. (2012). Viscosity behavior of high-concentration protein mixtures. J. Pharm. Sci. 101, 1012-1020.
He, F., Woods, C. E., Litowski, J. R., Roschen, L. A., Gadgil, H. S., Razinkov, V. I., & Kerwin, B. A. (2011). Effect of sugar molecules on the viscosity of high concentration monoclonal antibody solutions. Pharm. Res. 28, 1552-1560.
He, F., Woods, C. E., Trilisky, E., Bower, K. M., Litowski, J. R., Kerwin, B. A., Becker, G. W., Narhi, L. O., & Razinkov, V. I. (2010). Screening of monoclonal antibody formulations based on high-throughput thermostability and viscosity measurements: Design of experiment and statistical analysis. J. Pharm. Sci.
Leung, S. O., Goldenberg, D. M., Dion, A. S., Pellegrini, M. C., Shevitz, J., Shih, L. B., & Hansen, H. J. (1995). Construction and characterization of a humanized, internalizing, B-cell (CD22)-specific, leukemia/lymphoma antibody, LL2. Mol Immunol 32, 1413-1427.
Liu, J., Nguyen, M. D., Andya, J. D., & Shire, S. J. (2005). Reversible self-association increases the viscosity of a concentrated monoclonal antibody in aqueous solution. J. Pharm. Sci. 94, 1928-1940.
Sukumar, M., Doyle, B. L., Combs, J. L., & Pekar, A. H. (2004). Opalescent appearance of an IgG1 antibody at high concentrations and its relationship to noncovalent association. Pharm. Res. 21, 1087-1093.

1-14. (canceled)

15. A liquid pharmaceutical formulation comprising an antibody as active ingredient at a concentration of 200 to 400 mg/ml and acetate.

16. The liquid pharmaceutical formulation of claim 15, comprising acetate at a concentration of 20 to 150 mM.

17. The liquid pharmaceutical formulation of claim 15, comprising glycine at a concentration of 100 to 500 mM.

18. The liquid pharmaceutical formulation of claim 15, having an osmolality of 250 to 550 mOsm/kg.

19. The liquid pharmaceutical formulation of claim 15, having a viscosity of equal or less than 110 mPa s.

20. The liquid pharmaceutical formulation of claim 15, having a pH of 4.0 to 7.0.

21. The liquid pharmaceutical formulation of claim 15, comprising NaCl at a concentration of 0 to 100 mM.

22. The liquid pharmaceutical formulation of claim 15, comprising a surfactant.

23. The liquid pharmaceutical formulation of claim 22, wherein the surfactant is polysorbate 80.

24. The liquid pharmaceutical formulation of claim 22, wherein the surfactant is polysorbate 80 at a concentration of 0.001 to 0.03% w/v.

25. The liquid pharmaceutical formulation of claim 15, wherein said formulation does not comprise MgCl₂ or CaCl₂.

26. The liquid pharmaceutical formulation of claim 15, wherein the antibody is epratuzumab.

27. A container containing the liquid pharmaceutical formulation according to claim 15.

28. A kit containing the container of claim 27 and instructions for use.

29. A method for reducing the viscosity of a liquid pharmaceutical formulation containing a protein or an antibody, the method comprising:
a) providing the liquid pharmaceutical formulation, and
b) adding acetate to a final concentration of 20 to 150 mM,
wherein the viscosity of the liquid pharmaceutical formulation is reduced as compared to the same liquid pharmaceutical formulation without acetate.
